(19) United States
(12) Patent Application Publication
(10) Pub. No.: US 2006/0089387 A1
(43) Pub. Date: Apr. 27, 2006

(54) Stabilized pharmaceutical composition comprising antidiabetic agent

(76) Inventor: Le Huang, Shang Gao (CN)

Correspondence Address: Hui Min He-Huang, 53 Assabet Drive, Northborough, MA, 01532, US

(21) Appl. No.: 10/972,908

(22) Filed: Oct. 26, 2004

(51) Int. Cl. A61K 31/4439

(52) U.S. Cl. 514/340

(57) ABSTRACT

This invention discloses a stabilized pharmaceutical composition comprising an antidiabetic agent and a stabilizer. The preferred stabilizers are selected from the group consisting of ascorbic acid, malic acid, maleic acid, tartaric acid, furmaric acid, citric acid, or combinations thereof. The antidiabetic agent is selected from the group consisting of [(±)5-[[2-(5-ethyl-2-pyridinyl)ethoxyl]phenyl]methyl]-thiazolidine-2,4-dione and (±)5-[4-[2-(N-methyl-N-(2-pyridyl)amino)ethoxy]benzyl]thiazolidine-2,4-dione. This invention also discloses amorphous forms of said antidiabetic agents and a process of preparation thereof, and a method for medical treatment of diabetic mellitus using said pharmaceutical composition.

### FIELD OF THE INVENTION

The invention is directed to a stabilized pharmaceutical composition comprising antidiabetic agent such as free pioglitazone or free risoglitazone, and to a method of the treatment for diabetes mellitus using the composition. The present invention also relates to amorphous forms of free pioglitazone and free rosiglitazone, and to a process for the preparation of amorphous forms thereof.

### BACKGROUND OF THE INVENTION

Pioglitazone is the common name of [(±)5-[[2-(5-ethyl-2-pyridinyl) ethoxyl]phenyl]methyl]-thiazolidine-2,4-dione (hereinafter ‘free pioglitazone’), and its chemical structure is shown in Formula (1). Rosiglitazone is the common name of (±)5-[4-[2-(N-methyl-N-(2-pyridyl)amino)ethoxy]benzyl]thiazolidine-2,4-dione (hereinafter ‘free rosiglitazone’), and its chemical structure is shown in Formula (II). Free pioglitazone and free rosiglitazone belong to the thiazolidine-2,4-dione class of antidiabetic agents. Preparations of these compounds and the discovery of their antidiabetic properties were initially described in EP 0193256 and EP 0306228, respectively.

Free pioglitazone and free rosiglitazone are oral antidiabetic agents, which are believed to act by increasing insulin sensitivity and improving glycemic control while reducing circulating insulin. Commercial product AVANDIA contains rosiglitazone malate, the salt form of free rosiglitazone, and commercial product ACTOS contains pioglitazone hydrochloride, the salt form of free pioglitazone, are currently used for the management of non-insulin dependent type 2 diabetes mellitus. In addition, these antidiabetic agents in combination with insulin or other antidiabetic agents such as metformin hydrochloride can further increase their effects.

A number of European (EP), International (WO) and US patent applications 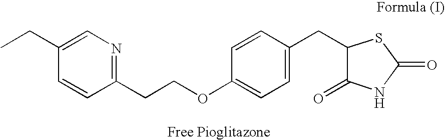 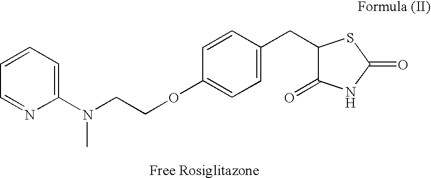
relate to free pioglitazone and its salts. European Patent Application (EP 0193256 or its equivalent U.S. Pat. No. 4,687,777) discloses the antidiabetic properties of [(±)5-[[2-(5-ethyl-2-pyridinyl)ethoxyl]phenyl]methyl]-thiazolidine-2,4-dione (free pioglitazone), its hydrochloride salt and sodium salt. WO02/088120 and WO04/024059 disclose a method to synthesize free pioglitazone and pioglitazone hydrochloride. WO02/28857 and U.S. Ser. No. 03/0,139,603 disclose two polymorphic forms (I and II) of pioglitazone hydrochloride salt. Above publications describe only the preparation of free pioglitazone as an intermediate or starting material for making the final salt form of free pioglitazone, and no details are provided as to the physical form and stability of free pioglitazone, and no details are provided as to the usage of free pioglitazone as pharmaceutical active agent for making pharmaceutical formulation. In other words, above patents and publications are silent on physiochemical properties of free pioglitazone, and silent on its usage as a pharmaceutical to make pharmaceutical compositions (e.g., solid dosage forms such as tablets and capsules) as well.

A number of European (EP), International (WO) patent applications and US patents relate to free rosiglitazone and its salts. European Patent Application (EP 0306228 or its equivalent U.S. Pat. No. 5,002,953) and U.S. Pat. No. 6,288,095 disclose certain thiazolidinedione derivatives, in particular, (±)5-[4-[2-(N-methyl-N-(2-pyridyl)amino) ethoxy]benzyl]thiazolidine-2,4-dione (free rosiglitazone), as having hypoglycaemic and hypolipidaemic activity. WO94/05659 (U.S. Pat. No. 5,741,803) discloses the preparation of certain salts of rosiglitazone, including the maleate salt at Example 1 thereof. WO0226737 (or U.S. Ser. No. 04/0,068,116) discloses polymorph Form-I and Form-II of rosiglitazone maleate. WO04/062667 discloses the preparation of an amorphous form of rosiglitazone maleate. WO03/050115 discloses the preparation and characterization of rosiglitazone citrate (citric acid addition salt). U.S. Pat. No. 6,664,278 teaches the preparation and characterization of hydrated form of rosiglitazone maleate. WO03/099337 discloses the preparation and characterization of host-guest complexes formed between free rosiglitazone or its salts and cyclodextrins. Above patents and publications, along with other publications related to acid addition or basic addition salts of free rosiglitazone, describe only the preparation of free rosiglitazone as an intermediate or starting material for making the final salt forms or host-guest complex of free rosiglitazone. WO03/045945 teaches the usage of free rosiglitazone in particulate form, which is less than 5 micrometers as pharmaceuticals, to make the aqueous dispersion and dosage forms. None of the above patents and publications provides any details on polymorph forms and stability of free rosiglitazone in pharmaceutical composition.

Although free pioglitazone as oral antidiabetic agent is generally taught, the prior art comprises numerous disclosures of salt formations of free pioglitazone, and teaches away from the utility of free pioglitazone as pharmaceutical products. Similarly, the prior art comprises numerous disclosures of salt formations of free rosiglitazone, and does not provide the details of polymorph forms and stability of free rosiglitazone in dosage forms. The main reason is that solid free pioglitazone and solid free rosiglitazone initially synthesized by the above applicants were characterized as unstable materials. In fact, both free pioglitazone and free rosiglitazone were found to undergo hydrolysis and oxidation in contact with water and in the presence of oxygen, to afford a thiol acetic acid, acyl amide and dimeric degradant, respectively (refer to Scheme-1 and Scheme-2). All of degradants were from thiazolidine-2,4-dione ring-opened degradants, and the thiol acetic acid served as an intermediate-like species, and further oxidized to the dimeric degradant.

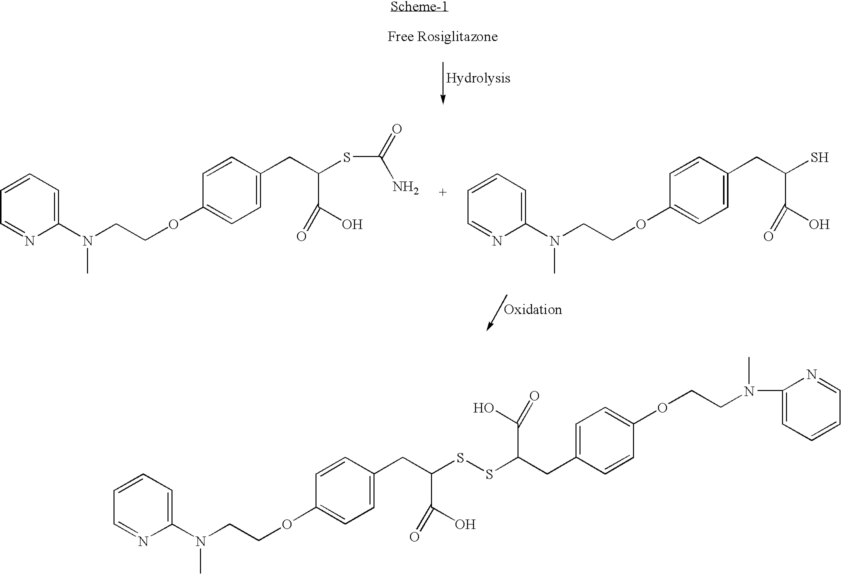

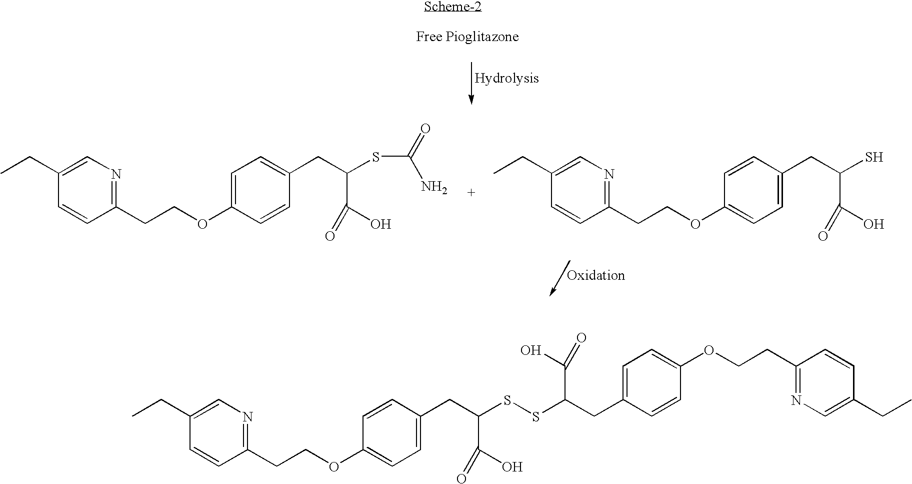

Surprisingly, the present inventor has found that free pioglitazone and free rosiglitazone, when mixed with stabilizers, are stable on long term storage and thus suitable to use in pharmaceutical compositions. In particular, pharmaceutical formulation comprising free pioglitazone and a pharmaceutically acceptable stabilizer has sufficient stability for long-term storage and use, and pharmaceutical formulation comprising free rosiglitazone and a pharmaceutically acceptable stabilizer also has sufficient stability for long-term storage and use. Such stability profiles were unexpected, based on the properties of free rosiglitazone and free pioglitazone materials which are susceptible to hydrolysis and oxidation. We discovered that free pioglitazone and free rosiglitazone in pharmaceutical compositions comprising a stabilizer do not undergo the decomposition reactions (hydrolysis and oxidation) described in Scheme-1 and Scheme-2 to any significant extent. Therefore, it is desirable to have a stable pharmaceutical composition comprising an antidiabetic agent and a stabilizer, which is equivalent to the commercial drug product such as ACTOS or AVANDIA.

### SUMMARY OF THE INVENTION

The present invention is based on the study of antidiabetic agents and the discovery of various physiochemical properties thereof. From a realization of these properties, the present invention provides a stable pharmaceutical composition comprising antidiabetic agent, especially a tablet or a capsule, and a process to stabilize antidiabetic agent in solid pharmaceutical compositions. The present invention further provides novel amorphous forms of antidiabetic agents, and a process for preparing amorphous forms thereof.

Accordingly, a first aspect of the present invention relates to a stabilized pharmaceutical composition comprising an antidiabetic agent and a pharmaceutically acceptable stabilizer in an effective stabilizing amount, wherein said pharmaceutical composition retains at least 95% weight/weight (hereinafter, w/w), preferably at least 96% w/w, of the initial potency of antidiabetic agent, and contains not more than 0.5% w/w single largest degradant and not more than 1.5% w/w total degradants after three (3) months storage at 40° C. and 75% relative humidity. The said antidiabetic agent is selected from the group consisting of free pioglitazone and free rosiglitazone. The said stabilizers are selected from the group consisting of ascorbic acid, citric acid, maleic acid, malic acid, fumaric acid, or tartaric acid.

Another aspect of the present invention is to provide a process comprising mixing active ingredient with a stabilizer, to stabilize free rosiglitazone or stabilize free pioglitazone in solid pharmaceutical compositions such as tablets or capsules.

A still further aspect of the present invention is to provide a novel amorphous form of free pioglitazone and a novel amorphous form of free rosiglitazone. These amorphous forms are suitable for use as pharmaceutical active agents.

Another aspect of the present invention relates to a process for the preparation of novel amorphous forms of free rosiglitazone and free pioglitazone.

Yet a further aspect of the present invention relates to a method of treating or preventing diabetes mellitus, particularly diabetes II mellitus, and conditions associated with diabetes mellitus and complication thereof in a mammal, which comprises administering an effective amount of free rosiglitazone or free pioglitazone to a patient in need thereof. Preferably free rosiglitazone or free pioglitazone is administered in the above-mentioned solid pharmaceutical compositions.

### BRIEF DESCRIPTION OF THE ACCOMPANYING DRAWINGS

FIG. 1 is X-ray powder diffraction pattern of novel amorphous form of free rosiglitazone

FIG. 2 is X-ray powder diffraction pattern of novel amorphous form of free pioglitazone

### DETAILED DESCRIPTION OF THE INVENTION

Accordingly, a first aspect of the present invention provides a stabilized pharmaceutical composition comprising an antidiabetic agent and a pharmaceutically acceptable stabilizer in an effective stabilizing amount, wherein said pharmaceutical composition retains at least 95% w/w, preferably at least 96%, of the initial potency of free pioglitazone or free rosiglitazone, and contains not more than 0.5% w/w single largest degradant and not more than 1.5% w/w, preferably not more than 1.0%, total degradants after three (3) months storage at 40° C. and 75% relative humidity, in which said antidiabetic agent is selected from the group consisting of free pioglitazone and free rosiglitazone, and in which said stabilizers are selected from the group consisting of ascorbic acid, citric acid, maleic acid, malic acid, fumaric acid, or tartaric acid.

In a preferred aspect, the present invention provides a solid pharmaceutical composition such as tablet or capsule comprising free rosiglitazone and an effective amount of a pharmaceutically acceptable stabilizer.

In a particular aspect, the present invention provides a solid pharmaceutical composition such as tablet or capsule comprising amorphous form of free rosiglitazone and an effective amount of a pharmaceutically acceptable stabilizer.

In another preferred aspect, the present invention provides a solid pharmaceutical composition such as tablet or capsule comprising free pioglitazone and an effective amount of a pharmaceutically acceptable stabilizer.

In a particular aspect, the present invention provides a solid pharmaceutical composition such as tablet or capsule comprising amorphous form of free pioglitazone and an effective amount of a pharmaceutically acceptable stabilizer.

An “antidiabetic agent” as used herein means the thiazolidinedione class of antidiabetic agents, preferably selected from the group consisting of free rosiglitazone and free pioglitazone.

A “stabilizer” as used herein means any pharmaceutically acceptable inactive component of the composition which inhibits (prevents or slows down) the decomposition of free rosiglitazone or free pioglitazone. The stabilizers, which are suitable for use in this invention, are those acids that have an aqueous solution pH of about 1.0 to 5.5 at an aqueous solution concentration of about 5% w/w.

The aqueous suspension pH of the stabilizers of this invention is determined by adding 3.5 grams of stabilizer to 60 grams of deionized water in a Pyrex® beaker. The resulting mixture is stirred for approximately 5 minutes, using a stir plate and a magnetic stir bar. The resulting suspension or dispersion is examined using a Corning® pH Meter Model 355. Suspensions are stirred with a magnetic stir bar during analysis. Measurements are performed in duplicate and the average thereof is used.

The stabilizers which meet the aforementioned pH range and are therefore included in the present invention are: ascorbic acid, citric acid, maleic acid, malic acid, fumaric acid, or tartaric acid, or combinations thereof. Ascorbic acid, citric acid and maleic acid are the preferred stabilizers for free rosiglitazone. Ascorbic acid and malic acid are the preferred stabilizers for free pioglitazone. Ascorbic acid is able to act as acidifier to inhibit or slow down the hydrolysis, and act as an antioxdidant to prevent or slow down the oxidation as well. Therefore ascorbic acid is the most preferred stabilizer for stabilizing free rosiglitazone and free pioglitazone in solid pharmaceutical compositions.

The amount of the stabilizer which should be used to achieve the results desired, for stabilization of free rosiglitazone or free pioglitazone in pharmaceutical solid dosage formulations, e.g., tablets and capsules is about 15%-75% w/w (or 0.50:1 to 1.50:1 molar ratio of stabilizer/active ingredient), preferably about 25%-60% w/w (or 0.8:1 to 1.2:1 molar ratio of stabilizer/active ingredient) and most preferably 28%-55% w/w (or 0.9:1 to 1.1:1 molar ratio of stabilizer/active ingredient). The amount percentage of stabilizer depends on the molarcular weight of the stabilizer, and the amount of stabilizer depends on the label strength of free rosiglitazone or free pioglitazone in solid pharmaceutical formulations, which can be readily determined by one skilled in art. For example, the amount is about 32-33% w/w for maleic acid, and about 37-38% w/w for malic acid, if the molar ratio of stabilizer/active ingredient is 1:1.

It should be understood that combinations of stabilizers may be used which meet the aforementioned pH conditions and to achieve the desired stability of the pharmaceutical product comprising free rosiglitazone and the pharmaceutical product comprising free pioglitazone.

The term “pharmaceutical composition” as used herein is intended to encompass a product including the active ingredient(s), pharmaceutically acceptable excipients that make up the carrier, as well as any product which results, directly or indirectly, from combination, or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, additional active ingredient(s), and pharmaceutically acceptable excipients including stabilizers.

A “free rosiglitazone” or “free pioglitazone” as used herein means any pharmaceutically acceptable physical forms, including amorphous forms, crystalline forms, solvates and hydrates of free rosiglitazone and free pioglitazone. The present invention intends to cover pharmaceutical compositions comprising amorphous, crystalline, solvate and hydrate forms of free rosiglitazone and a pharmaceutically acceptable stabilizer in an effective stabilizing amount. In addition, the present invention intends to cover pharmaceutical compositions comprising amorphous, crystalline, solvate and hydrate forms of free pioglitazone and a pharmaceutically acceptable stabilizer in an effective stabilizing amount.

Accordingly, free rosiglitazone in said pharmaceutical composition in the present invention includes amorphous form, crystalline form, or a mixture of amorphous form and crystalline form of free rosiglitazone. The amorphous form of free rosiglitazone disclosed in the present invention can be amorphous hydrate or amorphous anhydrous, which can be used in making said pharmaceutical composition.

Accordingly, free pioglitazone in said pharmaceutical composition in the present invention includes amorphous form, crystalline form, or a mixture of amorphous form and crystalline form of free pioglitazone. The amorphous form of free pioglitazone disclosed in the present invention can be amorphous hydrate or amorphous anhydrous, which can be used in making said pharmaceutical composition.

Anhydrous amorphous forms of free rosiglitazone and free pioglitazone are preferred active ingredient materials in pharmaceutical compositions of the present invention.

Furthermore, the term of “free rosiglitazone” as used in this invention also means rosiglitazone free base or rosiglitazone free acid, as shown in Formula (II). The term of “free pioglitazone” also means pioglitazone free base or pioglitazone free acid, as shown in Formula (I). In other words, free rosiglitazone or free pioglitazone of the present invention means that rosiglitazone and pioglitazone are in their original forms that are not treated (reacted) with acids or bases to form their respective salts.

Free rosiglitazone is a weak base and a weak acid (pKa1=6.8, pKa2=6.1). The nitrogen on the pyridyl ring in rosiglitazone acts as a weak base site, which can be protonated when reacted with a strong acid to form acid addition salt such as rosiglitazone maleate. In addition, the nitrogen-hydrogen bond in the thiazolidine-2,4-dione ring acts as a weak acid which can be ionized when reacted with a strong base to form base addition salt such as rosiglitazone sodium.

Similarly, free pioglitazone is a weak base and a weak acid as well (pKa1=6.5, pKa2=6.1). The nitrogen on the pyridinyl ring in pioglitazone acts as a weak base site, which can be protonated when reacted with a strong acid to form acid addition salt such as pioglitazone maleate. In addition, the nitrogen-hydrogen bond in the thiazolidine-2,4-dione ring acts as a weak acid which can be ionized when reacted with a strong base to form base addition salt such as pioglitazone sodium.

The total amount of inactive ingredient in the pharmaceutical composition, including the amount of stabilizer, is preferably more than 50% of the weight of free pioglitazone in the composition and less than 2,000% of the weight of free pioglitazone. The amount of stabilizer in terms of molar ratio of stabilizer/free pioglitazone may be from about 0.5:1 to 1.5:1 and is ideally about 0.8:1 to 1.2:1 in the pharmaceutical composition. Most preferably, the amount of stabilizer in terms of molar ratio is from about 0.9:1 to about 1.1:1. The suitable amount of stabilizer is based on the label strength of free pioglitazone and the molarcular weight of stabilizers in the pharmaceutical formulation in solid dosage form, and can be determined by one skilled in the art. For example, the amount of malic acid in pharmaceutical composition comprising free pioglitazone is 37% w/w, if the molar ratio of stabilizer/active ingredient is 1:1.

The total amount of inactive ingredient in the pharmaceutical composition, including the amount of stabilizer, is preferably more than 500% of the weight of free rosiglitazone in the composition and less than 20,000% of the weight of free rosiglitazone. The amount of stabilizer in terms of molar ratio of stabilizer/free rosiglitazone may be from about 0.5:1 to 1.5:1 and is ideally about 0.8:1 to about 1.2:1 in the pharmaceutical composition. Most preferably, the amount of stabilizer in terms of molar ratio is from about 0.9:1 to about 1.1:1. The suitable amount of stabilizer is based on the label strength of free rosiglitazone in the pharmaceutical formulation in solid dosage form and can be determined by one skilled in the art. For example, the amount of maleic acid in pharmaceutical composition comprising free rosiglitazone is 32.5% w/w if the molar ratio of stabilizer/active ingredient is 1:1.

The pharmaceutical composition of this invention preferably is one wherein the weight of free rosiglitazone in the tablet is Y and the amount of inactive ingredients is greater than about 500% of Y and less than about 20,000% of Y. The pharmaceutical composition of this invention most preferably is one wherein the weight of free rosiglitazone in the tablet is Y and the amount of inactive ingredients is greater than about 1,000% of Y and less than about 12,000% of Y. The tablets or capsules in this invention generally contain 1 mg to 15 mg of free rosiglitazone and usually contain about 1 mg, 2 mg, 4 mg, 8 mg, or 10 mg of free rosiglitazone.

The pharmaceutical composition of this invention preferably is one wherein the weight of free pioglitazone in the tablet is Y and the amount of inactive ingredients is greater than about 50% of Y and less than about 2,000% of Y. The pharmaceutical composition of this invention preferably is one wherein the weight of free pioglitazone in the tablet is Y and the amount of inactive ingredients is greater than about 400% of Y and less than about 1000% of Y. The tablets or capsules in this invention generally contain 5 mg to 60 mg of free pioglitazone and usually contain about 7.5 mg, 15 mg, 30 mg and 45 mg of free pioglitazone.

The stability of the pharmaceutical formulation was studied in accordance with industry standards by storage for three (3) months at about 40° C. and about 75% relative humidity (RH). Standard analytical procedures such as HPLC methods may be used to determine the amount of active ingredient and degradants remaining after storage. Pharmaceutical formulations containing stabilizers of the present invention stored under these conditions preferably retain at least 95% w/w, more preferably retain at least 96%, of the initial potency of free pioglitazone or free rosiglitazone, and contains not more than 0.5% w/w single largest degradant and preferably not more than 1.5% w/w, more preferably not more than 1.0%, total degradants after three (3) months storage under these conditions. In many instances, pharmaceutical formulations of the present invention retain more than 95% w/w and ideally retain more than 96% w/w of the initial potency of antidiabetic agents, and contain less than 0.5% w/w single largest degradant and less than than 1.0% w/w total degradants in the pharmaceutical composition after three (3) months storage under these conditions.

Furthermore, the pharmaceutical compositions prepared in accordance with this invention are solid in form, in which the amount of antidiabetic agents will be prevented from being reduced to not less than 95% w/w of its labeled strength, and preferably not less than 96% w/w of the labeled strength, while not more than 0.5% w/w single largest degradant and not more than 1.5% w/w total degradants of the labeled strength were formed after two years of storage under the usually encountered conditions (e.g., 25° C./60% relative humidity) in pharmacies and in medicine cabinets.

Pharmaceutical compositions of the present invention may optionally include any conventional ingredients for improving the physical properties, visual appearance or odor of the pharmaceutical. Examples include, but are not limited to, lubricants such as magnesium stearate or talc; binders such as starch, microcrystalline cellulose, hydroxypropyl cellulose, hydroxypropyl methylcellulose and polyvinylpyrrolidone; diluents such as microcrystalline cellulose and lactose; disintegrants such as sodium starch glycolate, crospovidone and croscarmellose sodium; and colorants.

The excipients are selected based on the desired physical aspects of the final form: e.g., obtaining a tablet with desired hardness and friability, being rapidly dispersible and easily swallowed, etc. The desired release rate of the active substance from the composition after its ingestion also plays a role in the choice of excipients. Preferred release rate is the rate comparable with commercially available rosiglitazone maleate tablets and pioglitazone hydrochloride tablets.

Surfactants or other solubiling agents may be used to aid the dissolution of drugs in the present invention. Suitable surfactant is sodium lauryl sulfate, and suitable solubilizing agents are PEG 400 and Poloxamer. The preferred solubilizing agent according to the present invention is Poloxamer.

According to a preferred aspect of the present invention, the water content of the excipients is very low. More specifically, the water content in the diluents is very low in order to minimize the water content of the pharmaceutical composition. Lactose is used in its anhydrous form. Furthermore, all excipients may be used in a dry form. Preferably all excipients used for the preparation of the composition should have water content below about 1%, preferably below about 0.5%, more preferred below about 0.1% (weight/weight).

According to another aspect, the present invention provides a process of stabilizing free pioglitazone or stabilize free rosiglitazone in solid pharmaceutical compositions to slow down the degradation thereof, by mixing free pioglitazone or free rosiglitazone with one or more stabilizers and other pharmaceutical excipients and followed by granulation in a high shear mixer. The granules can be filled into capsules or compressed into tablets or used in other pharmaceutical dosage forms.

In still another preferred aspect, pharmaceutical compositions of the present invention are prepared by wet granulation and followed by compression. For instance, the ingredients are screened and blended in an industrial blender such as a high shear blender. The blended material was then granulated with water. During the wet granulation, the contact time with water has to be very short, and granules are dried in a fluid bed drier. The concentrated granules and other excipients were further blended, and the mixture of ingredients are then compressed into tablets using, for instance, a Kikusui Libra® tablet compression machine.

In another preferred aspect, the manufacturing process applied is direct compression of tablets, wherein free rosiglitazone or free pioglitazone, one or more stabilizer and other excipients suitable for direct compression are mixed, followed by compression to make tablets.

Typically free rosiglitazone or free pioglitazone to be mixed is in the form of particles. The storage stability of the pharmaceutical composition of the present invention is enhanced, in general, by using larger particle sizes. Preferably the average particle size of free rosiglitazone or free pioglitazone is at least 1 microns, more preferably at least 5 microns, and most preferably in a range from 5 to 150 microns.

Tablets may be covered with a suitable coating. For example, the coating can be a moisture barrier to help with storage stability or a sustained or delayed release coating composition as are well known in the art.

Suitable package material for packing the pharmaceutical dosage forms are plastic or glass containers and blisters. Particularly blisters made from non-permeable materials (high density polyethylene or aluminum) are advantageous as they may contribute to decreasing the rate of formation of degradants.

In a preferred aspect, the present invention provides a process of stabilizing free rosiglitazone or free pioglitazone in a solid pharmaceutical composition so that at least 95% w/w of the initial potency of free rosiglitazone or free pioglitazone is retained in the undegraded form, and not more than 0.5% w/w single largest degradant or not more than 1.5% w/w total degradants are formed after storage for three (3) months at about 40° C. and 75% relative humidity, wherein said method comprises mixing free rosiglitazone or free pioglitazone with a stabilizer, the stabilizer being selected from the group consisting of ascorbic acid, citric acid, maleic acid, malic acid, fumaric acid, or tartaric acid.

For the poorly water-soluble drugs, it has been disclosed that the amorphous forms in a number of drugs exhibit different dissolution characteristics and in some cases different bioavailability patterns compared to crystalline forms [Konne T., Chem Pharm Bull, 38, 2003 (1990)]. For some therapeutic indications one bioavailability pattern may be favored over another. An amorphous form of cefuroxime axietil is typical example for exhibiting higher bioavailability than the crystalline forms. The amorphous forms of free rosiglitazone or free pioglitazone may also provide advantage in having an enhanced water solubility, dissolution rate or bioavailability since they are poorly water-soluble materials.

According to still another aspect, the present invention relates to free rosiglitazone in an amorphous form and free pioglitazone in an amorphous form.

In a preferred aspect, the present invention is directed to a novel amorphous form of free rosiglitazone.

In another preferred aspect, the present invention is directed to a novel amorphous form of free pioglitazone.

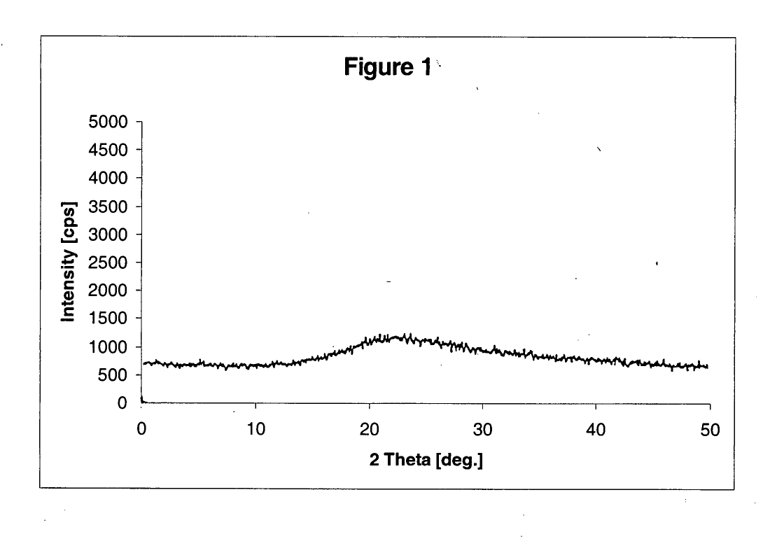

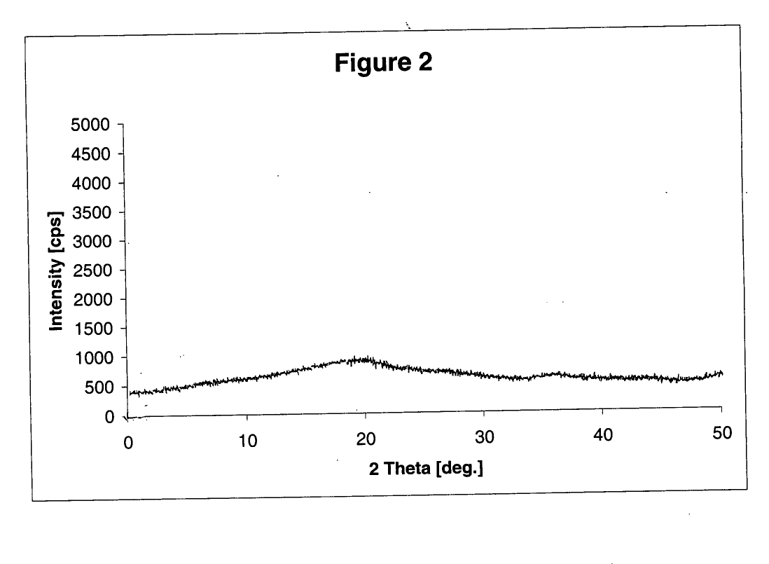

“Amorphous” means a solid without long-range crystalline order. Amorphous materials do not exhibit the three-dimensional long-range order found in crystalline materials but are structurally more similar to liquids where the arrangement of molarcules is random. Amorphous solids are not crystalline and therefore do not give a definitive x-ray diffraction pattern (XPD). A sample of an XPD spectrum of amorphous form of free rosiglitazone by this inventor is shown in FIG. 1, and a sample of an XPD spectrum of amorphous form of free pioglitazone by this inventor is shown in FIG. 2. As seen therefrom, the XPD patterns are highly characteristics of amorphous solids.

Amorphous form of free rosiglitazone or free pioglitazone in accordance with the present invention preferably contains less than about 15% crystalline forms of free rosiglitazone or free pioglitazone, and more preferably is essentially free of crystalline forms of free rosiglitazone or free pioglitazone. “Essentially free of crystalline forms” means that no crystalline forms can be detected within the limits of a powder X-ray diffraction instrument.

The present invention also includes a composition of solid free rosiglitazone or solid free pioglitazone wherein at least 85% of the total weight of free rosiglitazone or free pioglitazone is in the amorphous form. In a preferred form of this composition, the solid free rosiglitazone or solid free pioglitazone is suitable for use as a bulk active ingredient in formulating pharmaceutical products. The remainder of the solid free rosiglitazone or solid free pioglitazone in the composition, i.e., 15% or less of the total weight of free rosiglitazone or free pioglitazone, may be other forms of free rosiglitazone or free pioglitazone, e.g. crystalline forms or polymorphs.

The present invention further includes the composition that may include at least 95% of amorphous form of free rosiglitazone or free pioglitazone with respect to total weight of solid free rosiglitazone or solid free pioglitazone in the composition. Preferably, the composition may include at least 99% of amorphous form of free rosiglitazone or free pioglitazone with respect to total weight of solid free rosiglitazone or solid free pioglitazone in the composition. Most preferably, the composition is substantially free of any forms of free rosiglitazone or free pioglitazone other than its amorphous form.

The preferred method of differentiating amorphous form of free rosiglitazone or free pioglitazone from other crystalline and non-crystalline forms of free rosiglitazone or free pioglitazone is X-ray powder diffraction (XPD). The XPD pattern of pure amorphous free rosiglitazone or free pioglitazone, as illustrated in FIG. 1 and FIG. 2, can be seen to lack discernible acute peaks. Thus, amorphous form of free rosiglitazone or free pioglitazone, according to the present invention, is characterized in providing an X-ray powder diffraction pattern containing one or more broad diffuse halos having very low counts (i.e. see FIG. 1 and FIG. 2) in contrast to the sharp diffraction peaks characteristic of crystalline materials. Of course it will be appreciated that a mixture comprising detectable amounts of both crystalline and amorphous form of free rosiglitazone or free pioglitazone will exhibit both the characteristic sharp peaks and the diffuse halo(s) on XPD. This will be evident by an increase in the baseline and also a reduction in crystalline peak intensities.

According to a further aspect of the present invention, there is provided a process for preparation of an amorphous form of free pioglitazone, including the steps of dissolving free pioglitazone in a solvent to form a solution, distilling the solvent from the solution to dryness, and isolating the solid residue to afford amorphous form of free pioglitazone.

According to another aspect of the present invention, there is provided a process for preparation of an amorphous form of free rosiglitazone, including the steps of dissolving free rosiglitazone in a solvent to form a solution, distilling the solvent from the solution to dryness, and isolating the solid residue to afford amorphous form of free rosiglitazone.

In a first step of the process, free rosiglitazone or free pioglitazone is preferably dissolved in an alcoholic solvent, acetinotrile or tetrahydrofuran; more preferably dissolved in a straight or branched chain C₁-C₄ alcohol solvent, acetinotrile or tetrahydrofuran, and most preferably dissolved in methanol, acetonitrile or tetrahydrofuran to form a solution. Free rosiglitazone or free pioglitazone is soluble in methanol, acetonitrile or tetrahydrofuran, allowing the complete dissolution of these compounds at ambient temperature or at boiling point of the solvent.

In particular, free rosiglitazone or free pioglitazone is soluble in methanol, allowing the complete dissolution of these compounds in methanol at an increased temperature (e.g., 50° C.) with a concentration of 500 mg per 100 milliliter (ml). The use of a relatively concentrated solution, e.g. 500 mg per 100 ml is therefore preferred for methanol.

Similarly, free rosiglitazone or free pioglitazone is soluble in ethanol, allowing the complete dissolution of these compounds in ethanol at an increased temperature (50° C.) with a concentration of 500 mg per 100 ml. The use of a relatively concentrated solution, e.g. about 500 mg per 100 ml is therefore preferred for ethanol.

Furthermore, free rosiglitazone or free pioglitazone is soluble in acetonitrile, allowing the complete dissolution of these compounds in acetonitrile at elevated temperature (e.g., 50° C.) with a concentration of 500 mg per 100 ml. The use of a relatively concentrated solution, e.g. about 500 mg per 100 ml is therefore preferred for acetonitrile.

In addition, free rosiglitazone or free pioglitazone is soluble in tetrahydrofuran, allowing the complete dissolution of these compounds in tetrahydrofuran at ambient temperature (e.g., 25° C.) with a concentration of 500 mg per 50 ml. The use of a relatively concentrated solution, e.g. about 500 mg per 50 ml is therefore preferred for tetrahydrofuran.

In a second step of the process, using conventional distillation methods, the solvent is removed from the solution to dryness, thereby leaving a solid residue containing amorphous form of free rosiglitazone or amorphous form of free pioglitazone.

The distillation process can be preformed at atmospheric pressure or reduced pressure. Preferably the solvent is removed at a pressure of about 760 mm Hg or less, more preferably at about 400 mm Hg or less, more preferably at about 80 mm Hg or less, and most preferably from about 30 to about 80 mm Hg.

The distilling of the solvent from free rosiglitazone solution or free pioglitazone solution may be carried out at an increased temperature, preferably at reflux temperature, and/or at reduced pressure. Preferably, the removal of the solvent may be carried out preferably below 75° C., more preferably at about 40° C.-75° C., yet more preferably at about 55° C.-70° C. The solid residue obtained after solvent removal may be isolated and dried using conventional methods. The advantages of the process include simplicity and suitability for commercial use.

The straight or branched chain C₁-C₄ alcohol solvents are selected from the group consisting of methanol, ethanol, n-propanol, isopropanol or branched-chain butanols. It is preferred to use methanol or ethanol, or a mixture of methanol and ethanol. The process may also be carried out by using a mixture of two or more other alcohol solvents.

The novel amorphous forms of free rosiglitazone and free pioglitazone obtained in above procedures can be amorphous anhydrous and amorphous hydrate. The current invention intends to cover both anhydrous and hydrate amorphous forms of free rosiglitazone. The current invention also intends to cover both anhydrous and hydrate amorphous forms of free pioglitazone.

It has been unexpectedly found that uniformly anhydrous or hydrate amorphous forms of free rosiglitazone can be obtained in simple and reproducible processes as described above.

Furthermore, it has been unexpectedly found that uniformly anhydrous or hydrate amorphous forms of free pioglitazone can be obtained in simple and reproducible processes as described above.

Amorphous form of free rosiglitazone or amorphous form of free pioglitazone prepared according to the processes of the present invention may be characterized by its x-ray powder diffraction pattern, as shown in the accompanied drawings of FIG. 1 and FIG. 2. The X-ray powder diffraction pattern (FIG. 1 and FIG. 2) shows no peaks which are characteristic of amorphous forms of free rosiglitazone and free pioglitazone, thus demonstrating the amorphous nature of the product. The amorphous forms of these two compounds shown in FIG. 1 and FIG. 2 are produced by above described process.

Free rosiglitazone and free pioglitazone can be made by any of the known techniques set forth in the prior art. It is desirable that free rosiglitazone active or free pioglitazone active be substantially pure. For example, the content of impurities that can be produced during the synthesis should be limited, preferably to less than 2% w/w, although such purity is not required for the present invention.

The starting materials for preparing amorphous form of free rosiglitazone can be crude or pure crystalline material of free rosiglitazone which can be prepared from crude or pure rosiglitazone maleate salt. The crystalline material of free rosiglitazone may also be prepared according to known procedures such as those described in U.S. Pat. Nos. 5,002,953; 5,646,169, 5,741,803 and 6,288,095. Rosiglitazone maleate may be prepared according to known procedures such as those disclosed in U.S. Pat. Nos. 5,002,953; 5,646,169; 5,741,803 and 6,288,095.

The starting materials for preparing amorphous form of free pioglitazone can be crude or pure crystalline material of free pioglitazone which can be prepared from crude or pure pioglitazone hydrochloride salt. The crude or pure crystalline material of free pioglitazone may also be prepared according to known procedures such as those described in U.S. Pat. Nos. 4,687,777; 5,585,495 and WO02/088120. Pioglitazone hydrochloride may be prepared according to known procedures such as those disclosed in U.S. Pat. Nos. 4,687,777; 5,585,495 and WO02/088120.

A further aspect of the invention relates to a method of treating or preventing diabetes mellitus, particularly type 2 diabetes mellitus, and prophylaxis of conditions associated with diabetes mellitus and complication thereof in a mammal, comprising administering to a patient in need of such treatment an effective amount of a pharmaceutical composition comprising free rosiglitazone and a pharmaceutically acceptable stabilizer, and comprising administering to a patient in need of such treatment an effective amount of a pharmaceutical composition comprising free pioglitazone and a pharmaceutically acceptable stabilizer. Still conditions associated with diabetes include hyperglycaemia and insulin resistance, especially acquired insulin resistance and obesity. Further conditions associated with diabetes include hypertension, cardiovascular disease, especially arteriosclerosis, and anorexia bulimia.

The complications of conditions associated with diabetes mellitus encompassed herein includes renal disease, especially renal disease associated with the development of Type 2 diabetes including diabetic nephropathy, glomerular sclerosis, nephritic syndrome, hypertensive nephrosclerosis and end stage renal disease.

When used herein, the expression “prophylaxis of conditions associated with diabetes mellitus” includes the treatment of conditions such as insulin resistance, impaired glucose tolerance, hyperinsulinaemia and gestational diabetes.

The invention also provides a use of free rosiglitazone or free pioglitazone along with one or more additional agents. Since free rosiglitazone or free pioglitazone is an insulin sensitivity enhancer, a combination therapy with one or more other agents may be particularly desirable. The additional agents that can be used with free rosiglitazone or free pioglitazone, for example, include metformin, sulfonylurea, their pharmaceutically acceptable salts, insulin, and combinations thereof. Preferably, free pioglitazone or free rosiglitazone is combined with metformin hydrochloride. The additional agent may be combined with free rosiglitazone or free pioglitazone to form a single pharmaceutical dosage form, for example, such as tablets or capsules or may be prepared as a separate pharmaceutical dosage form, which can be administered to a patient along with the pharmaceutical dosage form of free rosiglitazone or free pioglitazone at the same time or with a time interval depending upon the patients conditions, and additional agents being used for the combination therapy. The pharmaceutical dosage form may include any forms of drug, which are suitable transport the therapeutic agents into body as noted hereinabove.

In a preferred aspect of the present invention, one example of such pharmaceutical dosage form of the combination therapy is a tablet, which may contain free rosiglitazone and metformin hydrochloride equivalent to: 1 mg free rosiglitazone with 500 mg metformin hydrochloride, 2 mg free rosiglitazone with 500 mg metformin hydrochloride, or 4 mg free rosiglitazone with 500 mg metformin hydrochloride in addition to inactive ingredients such as lactose monohydrate, magnesium stearate, microcrystalline cellulose, pregelatinized starch, sodium starch glycolate, titanium dioxide and one or more of red and yellow iron oxides.

In another preferred aspect of the present invention, one example of such pharmaceutical dosage form of the combination therapy is a tablet, which may contain free pioglitazone and metformin hydrochloride equivalent to: 7.5 mg free pioglitazone with 500 mg metformin hydrochloride, 15 mg free pioglitazone with 500 mg metformin hydrochloride, 30 mg free pioglitazone with 500 mg metformin hydrochloride, or 45 mg free pioglitazone with 500 mg metformin hydrochloride in addition to inactive ingredients such as lactose monohydrate, magnesium stearate, microcrystalline cellulose, pregelatinized starch, sodium starch glycolate, titanium dioxide and one or more of red and yellow iron oxides.

### EXAMPLES

The following examples are illustrative, but are not limiting of the present invention. Throughout the examples, NF and USP are designations for standards published in the National Formulary and U.S. Pharmacopoeia, respectively.

### Example 1

### Formulation of Amorphous Form of Free Rosiglitazone Tablets Comprising Maleic Acid

Formulation described in examples was calculated for a batch size which yields 7850 tablets. There were three major steps involved in manufacturing the tablets: (A) preparation of free rosiglitazone granular concentrate; (B) preparation of free rosiglitazone tablet core; (C) coating the tablet core. The amount of each ingredient included in the formulation is shown in Table 1 (quantity in gram) and Table 2.

TABLE 1
% Composition of Free Rosiglitazone
(10%, w/w) Granular Concentrate

| | Example number | | | | | |
|---|---|---|---|---|---|---|
| | 1 | 2 | 3-A | 4 | 5 | 3-B |
| Free rosiglitazone | 31.4 | 31.4 | 31.4 | 31.4 | 31.4 | 31.4 |
| | (a) | (b) | (a) | (a) | (a) | (a) |
| Ascorbic acid | | | 15.5 | 7.5 | | 15.5 |
| Maleic acid | 10.2 | 10.2 | | 8.0 | | |
| Lactose anhydrous | 191.8 | 191.8 | 188.1 | 188.1 | | |
| Lactose monohydrate | | | | | 203.6 | 188.1 |
| Sodium starch glycolate | 10 | 10 | 10 | 10 | 10 | 10 |
| Pregelatinized starch | 15.6 | 15.6 | 14 | 14 | 14 | 14 |
| Microcrystalline | 55 | 55 | 55 | 55 | 55 | 55 |
| cellulose | | | | | | |
| Purified water* | | | | | | |

(a): Amorphous form of free rosiglitazone
(b): Crystalline form of free rosiglitazone
*Water was removed during the process

A: Preparation of Free Rosiglitazone Granular Concentrate

The following ingredients (quantity in gram) were sifted through a clean screen (typically 0.066″): lactose anhydrous or lactose monohydrate, maleic acid, pregelatinized starch, sodium starch glycolate and microcrystalline cellulose.

The screened materials were transferred into a high shear (high-energy) mixer and blended for ten (10) minutes at 100 rpm. The blended material was granulated with purified water. The wet granules were passed through a screen (typically 0.132″), and dried in a fluid bed drier until loss on drying is less than 0.2-0.5% w/w.

The dried granules were passed a screen (typically 0.039″) and blended using a tumble blender for 10 minutes at 12 rpm.

B: Preparation of Free Rosiglitazone Tablet Core

The concentrated granules are placed into a tumble blender. About two thirds of the lactose is screened and added to the blender, and blended for ten (10) minutes. The microcrystalline cellulose, sodium starch glycollate, magnesium stearate and remaining lactose are screened and added to the blender. The mixtures are blended together for ten (10) minutes. The blended material was compressed on a Kikusui Libra tablet compression machine to a target weight of 150 mg for the 2 mg, 4 mg, or 8 mg tablets.

TABLE 2
% Composition of Free Rosiglitazone Tablet
Core (quantity, mg per tablet)

| | Example number | | | |
|---|---|---|---|---|
| | 1, 2, 3-A, | | | |
| | 4 | 1, 3-A, 4 | 1, 3-A, 4 | 5, 3-B |
| Dosage strength | 4 mg | 2 mg | 8 mg | 4 mg |
| Free rosiglitazone | 40.0 | 20.0 | 80.0 | 40.0 |
| concentrate granules | | | | |
| Lactose anhydrous | 85.94 | 105.94 | 45.94 | |
| Lactose | | | | 85.94 |
| monohydrate | | | | |
| Microcrystalline | 23.31 | 23.31 | 13.31 | 23.31 |
| cellulose | | | | |
| Magnesium stearate | 0.75 | 0.75 | 0.75 | 0.75 |
| Total weight | 150.0 | 150.0 | 150.0 | 150.0 |
| Coating material | 4.0 | 4.0 | 4.0 | 4.0 |
| Total weight of | 154.0 | 154.0 | 154.0 | 154.0 |
| coated tablet | | | | |

C: Preparation of Free Rosiglitazone Coated Tablet

The tablet cores are then transferred to a tablet-coating machine (pan coater). The tablet bed was pre-heated with warm air (approximately 60° C.). The pan speed was adjusted to 5-9 RPM before starting the spray cycle. The spray cycle was activated. The exhaust temperature was maintained between 40° C. and 50° C. throughout the cycle. After the proper amount of solution was applied, the coated tablets were dried for approximately two (2) minutes. Steps were repeated for all pans to coat all tablets in the batch and film coated until the tablet weight has increased by 2.0% to 3.5%. All tablets were packaged in plastic bottles with desiccants, and the bottles were heat sealed, then placed under the stress condition.

### Example 2

### Formulation of Crystalline Form of Free Rosiglitazone Tablets Comprising Maleic Acid

The 4 mg tablets were manufactured according to the procedure outlined in Example 1 except crystalline form of free rosiglitazone was used as an active ingredient in the formulation. The amount of each excipient is shown in Table 1 (quantity in gram) and Table 2.

### Examples 3-A and 3-B

### Formulation of Amorphous Form of Free Rosiglitazone Tablets Comprising Ascorbic Acid

The 2 mg, 4 mg, or 8 mg tablets were manufactured for Example 3-A, and the 4 mg tablets made for Example 3-B according to the procedure outlined in Example 1 except ascorbic acid was used as a stabilizer in the formulation. Table 1 (quantity in gram) and Table 2 outlines the quantity of each ingredient in the formulation containing 1:1 molar ratio of free rosiglitazone and ascorbic acid. Example 3-A uses lactose anhydrous and Example 3-B uses lactose monohydrate.

### Example 4

### Formulation of Amorphous Form of Free Rosiglitazone Tablets Comprising Ascorbic Acid and Maleic Acid

The 2 mg, 4 mg, or 8 mg tablets were manufactured according to the procedure outlined in Example 1 except ascorbic acid and maleic acid were used as stabilizers in the composition. Table 1 (quantity in gram) and Table 2 outline the quantity of each ingredient in the formulation.

### Example 5

### Formulation of Amorphous Form of Free Rosiglitazone Tablets Without Stabilizer

The 4 mg tablets were manufactured according to the procedure outlined in Example 1 except lactose monohydrate and no stabilizer were used in the composition. Table 1 (quantity in gram) and Table 2 outline the quantity of each ingredient in the formulation.

### Example 6

### Formulation of Amorphous Form of Free Pioglitazone Tablets Comprising Malic Acid

Formulation described in this example was calculated for a batch size which yields 4173 tablets. There were three major steps involved in manufacturing the tablets: (A) preparation of free pioglitazone granular concentrate; (B) preparation of free pioglitazone tablet core; (C) coating the tablet core. The amount of each ingredient included in the formulation is shown in Table 3 (quantity in gram) and Table 4.

A: Preparation of Free Pioglitazone Granular Concentrate

The following ingredients (quantity in gram) were sifted through a clean screen (typically 0.066″): lactose anhydrous or lactose monohydrate, malic acid, pregelatinized starch, sodium starch glycolate and microcrystalline cellulose, NF.

The screened materials were transferred into a high shear (high-energy) mixer blender and blended for ten (10) minutes at 100 rpm. The blended material was granulated with purified water. The wet granules were passed through a screen (typically 0.132″), and dried in a fluid bed drier until loss on drying is 0.2-0.5%.

The dried granules were passed a screen (typically 0.039″) and blended using a tumble blender for 10 minutes at 12 rpm.

TABLE 3
% Composition of Free Pioglitazone
(30%, w/w) Granular Concentrate

| | Example number | | | | | |
|---|---|---|---|---|---|---|
| | 6 | 7 | 8-A | 9 | 10 | 8-B |
| Free pioglitazone | 125.2 | 125.2 | 125.2 | 125.2 | 125.2 | 125.2 |
| | (a) | (b) | (a) | (a) | (a) | (a) |
| Ascorbic acid | | | 61.8 | 31.8 | | 61.8 |
| Malic acid | 45.9 | 45.9 | | 30.0 | | |
| Lactose anhydrous | 180.0 | 180.0 | 174.0 | 174.0 | | |
| Lactose monohydrate | | | | | 235.8 | 174.0 |
| Sodium starch glycolate | 10 | 10 | 10 | 10 | 10 | 10 |
| Pregelatinized starch | 10 | 10 | 14 | 14 | 14 | 14 |
| Microcrystalline | 45.9 | 45.9 | 35 | 35 | 35 | 35 |
| cellulose | | | | | | |
| Purified water* | | | | | | |

(a): Amorphous form of free pioglitazone
(b): Crystalline form of free pioglitazone
*Water was removed during the process

B: Preparation of Free Pioglitazone Tablet Core

The concentrated granules are placed into a tumble blender. About two thirds of the lactose is screened and added to the blender, and blended for ten (10) minutes. The microcrystalline cellulose, sodium starch glycollate, magnesium stearate and remaining lactose are screened and added to the blender. The mixtures are blended together for ten (10) minuets. The blended material was compressed on a Kikusui Libra tablet compression machine to a target weight of 300 mg for the 30 mg free pioglitazone tablets.

C: Preparation of Free Pioglitazone Coated Tablets

The tablet cores are then transferred to a tablet-coating machine (pan coater). The tablet bed was pre-heated with warm air (approximately 60° C.). The pan speed was adjusted to 5-9 RPM before starting the spray cycle. The spray cycle was activated. The exhaust

TABLE 4
% Composition of Free Pioglitazone Tablet
Core (quantity, mg per tablet)

| | Example number | | | |
|---|---|---|---|---|
| | 6, 7, 8-A, 9 | 6, 8-A, 9 | 6, 8-A, 9 | 8-B, 10 |
| Dosage strength | 15 mg | 30 mg | 45 mg | 30 mg |
| Free pioglitazone | 50.0 | 100 | 150.0 | 100 |
| concentrate granules | | | | |
| Lactose anhydrous | 190 | 160 | 110 | |
| Lactose | | | | 160 |
| monohydrate | | | | |
| Microcrystalline | 35 | 23.5 | 23.5 | 23.5 |
| cellulose | | | | |
| Sodium starch glycolate | 23.5 | 15 | 15 | 15 |
| Magnesium stearate | 1.5 | 1.5 | 1.5 | 1.5 |
| Total weight of | 300 | 300 | 300 | 300 |
| tablet core | | | | |
| Coating material | 8 | 8 | 8 | 8 |
| Total weight of | 308 | 308 | 308 | 308 |
| coated tablet | | | | |

temperature was maintained between 40° C. and 50° C. throughout the cycle. After the proper amount of solution was applied, the coated tablets were dried for approximately two (2) minutes. Steps were repeated for all pans to coat all tablets in the batch and film coated until the tablet weight has increased by 2.0% to 3.5%. All tablets were packaged in plastic bottles with desiccants, and the bottles were heat sealed, then placed under the stress condition.

### Example 7

### Formulation of Crystalline Form of Free Pioglitazone Tablets Comprising Malic Acid

The 15 mg tablets were manufactured according to the procedure outlined in Example 6 except crystalline form of free pioglitazone was used as an active ingredient in the formulation. The amount of each excipient is shown in Table 3 (quantity in gram) and Table 4.

### Example 8-A and 8-B

### Formulation of Amorphous Form of Free Pioglitazone Tablets Comprising Ascorbic Acid

The 15 mg, 30 mg, or 45 mg tablets were manufactured for Example 8-A, and the 30 mg tablets made for Example 8-B according to the procedure outlined in Example 6 except ascorbic acid was used as a stabilizer in the composition. Table 3 (quantity in gram) and Table 4 outlines the quantity of each ingredient in the formulation containing 1:1 molar ratio of free pioglitazone and ascorbic acid. Example 8-A uses lactose anhydrous and Example 8-B uses lactose monohydrate.

### Example 9

### Formulation of Amorphous Form of Free Pioglitazone Tablets Comprising Ascorbic Acid and Malic Acid

The 15 mg, 30 mg, or 45 mg tablets were manufactured according to the procedure outlined in Example 6 except ascorbic acid and maleic acid were used as stabilizers in the composition. Table 3 (quantity in gram) and Table 4 outline the quantity of each ingredient in the formulation.

### Example 10

### Formulation of Amorphous Form of Free Pioglitazone Tablets Without Stabilizer

The 30 mg tablets were manufactured according to the procedure outlined in Example 6 except lactose monohydrate and no stabilizer were used in the composition. Table 3 and Table 4 outline the quantity of each ingredient in the formulation.

### Example 11

### Stability Studies on Free Rosiglitazone Tablets and Free Pioglitazone Tablets

Stability studies on batches produced in Examples 1-10 were performed in a thermostated chamber adjusted to 40° C. and 75% of relative humidity in package material of HDPE bottles. Assay of the active substance and of the content of degradants was performed by HPLC method, using reference materials of free rosiglitazone or free pioglitazone and their major degradants. The content of other detected impurities or degradation products was calculated by internal area normalization. In Tables 5-8, the assay of the active substance and degradants are expressed in percentage.

TABLE 5
Stability studies of free rosiglitazone at 40° C./75% RH

| | Example number | | | | | |
|---|---|---|---|---|---|---|
| | 2 | 5 (3-B) | | 1 | | |
| Dosage strength | 4 mg | 4 mg | (4 mg) | 2 mg | 4 mg | 8 mg |
| t = 0 months | | | | | | |
| Assay (potency, %) | 99.1 | 99.0 | (99.1) | 99.5 | 99.2 | 99.1 |
| Largest degradant (%) | <0.10 | <0.10 | (<0.10) | <0.10 | <0.10 | <0.10 |
| Total degradants (%) | <0.10 | 0.14 | (0.10) | <0.10 | <0.10 | <0.10 |
| T = 3 months | | | | | | |
| Assay (potency, %) | 96.9 | 94.3 | (96.3) | 96.5 | 96.6 | 96.8 |
| Largest degradant (%) | 0.32 | 0.67 | (0.38) | 0.36 | 0.35 | 0.33 |
| Total degradants (%) | 0.65 | 1.65 | (0.75) | 0.69 | 0.65 | 0.64 |

The stability data from Table 5 and Table 7 indicated that about 95% w/w of the initial potency of free pioglitazone or free rosiglitazone was retained after three (3) months at 40° C./75% RH in pharmaceutical composition containing antidiabetic agent and no stabilizer. At mean time, the single largest degradant was more than 0.6% w/w, and the total degradants were more than 1.6% w/w in the composition.

TABLE 6
Stability studies of free rosiglitazone at 40° C./75% RH

| | Example number | | | | | |
|---|---|---|---|---|---|---|
| | 3-A | | | 4 | | |
| Dosage strength | 2 mg | 4 mg | 8 mg | 2 mg | 4 mg | 8 mg |
| t = 0 months | | | | | | |
| Assay (potency, %) | 98.8 | 98.9 | 99.2 | 99.0 | 99.3 | 99.1 |
| Largest degradant (%) | <0.10 | <0.10 | <0.10 | <0.10 | <0.10 | <0.10 |
| Total degradants (%) | <0.10 | <0.10 | <0.10 | <0.10 | <0.10 | <0.10 |
| T = 3 months | | | | | | |
| Assay (potency, %) | 97.1 | 97.3 | 97.6 | 97.2 | 97.5 | 97.4 |
| Largest degradant | 0.30 | 0.28 | 0.26 | 0.29 | 0.27 | 0.27 |
| Total degradants (%) | 0.63 | 0.59 | 0.58 | 0.62 | 0.57 | 0.58 |

TABLE 7
Stability studies of free pioglitazone at 40° C./75% RH

| | Example number | | | | | |
|---|---|---|---|---|---|---|
| | 7 | 10 (8-B) | | 6 | | |
| Dosage strength | 15 | 30 | (30 | 15 | 30 | 45 |
| | mg | mg | mg) | mg | mg | mg |
| t = 0 months | | | | | | |
| Assay (potency, %) | 99.1 | 98.9 | (99.0) | 99.3 | 99.0 | 99.2 |
| Largest degradant (%) | <0.10 | <0.10 | (<0.10) | <0.10 | <0.10 | <0.10 |
| Total degradants (%) | <0.10 | 0.11 | (<0.10) | <0.10 | <0.10 | <0.10 |
| T = 3 months | | | | | | |
| Assay (potency, %) | 97.2 | 94.0 | (96.6) | 96.9 | 97.0 | 97.4 |
| Largest degradant (%) | 0.29 | 0.65 | (0.35) | 0.32 | 0.30 | 0.31 |
| Total degradants (%) | 0.62 | 1.62 | (0.72) | 0.65 | 0.62 | 0.59 |

The stability data from Table 5-8 indicated that near or above 97.5% w/w of the initial potency of free pioglitazone or free rosiglitazone was retained after three (3) months at 40° C./75% RH in pharmaceutical composition containing antidiabetic agent and a stabilizer. In addition, not more than 1.0% w/w total degradants and not more than 0.5% w/w single largest degradant of the labeled claim were formed after three (3) months at 40° C./75% RH in the composition.

TABLE 8
Stability studies of free pioglitazone at 40° C./75% RH

| | Example number | | | | | |
|---|---|---|---|---|---|---|
| | 8-A | | | 9 | | |
| Dosage strength | 15 | 30 | 45 | 15 | 30 | 45 |
| | mg | mg | mg | mg | mg | mg |
| t = 0 months | | | | | | |
| Assay (potency, %) | 99.0 | 99.2 | 99.1 | 99.4 | 99.0 | 99.5 |
| Largest degradant (%) | <0.10 | <0.10 | <0.10 | <0.10 | <0.10 | <0.10 |
| Total degradants (%) | <0.10 | <0.10 | <0.10 | <0.10 | <0.10 | <0.10 |
| T = 3 months | | | | | | |
| Assay (potency, %) | 97.5 | 97.2 | 97.6 | 97.1 | 97.3 | 97.8 |
| Largest degradant | 0.29 | 0.26 | 0.25 | 0.27 | 0.26 | 0.25 |
| Total degdadants (%) | 0.58 | 0.57 | 0.55 | 0.56 | 0.59 | 0.55 |

Therefore, these stability data clearly demonstrated that ascorbic acid, malic acid or maleic acid are suitable and effective stabilizers for retaining potency and inhibiting the degradation of free pioglitazone or free rosiglitazone in solid pharmaceutical composition such as tablets.

### Example 12

### Preparation of Amorphous Form of (±)5-[4-[2-(N-methyl-N-(2-pyridyl)amino) ethoxy]benzyl]thiazolidine-2,4-dione (Free Rosiglitazone)

Crystalline material of free rosiglitazone (500 mg, prepared according to Example 30 of U.S. Pat. No. 5,002,953) was dissolved in methanol (100 ml) under stirring at 50° C., and the solution is heated to boiling (about 70 to 75° C.) until the material goes into solution. The solvent was completely distilled off under reduced pressure at 60° C. to 70° C. and the solid residue was isolated and dried in vacuum at 40° C. Thus 450 mg of amorphous form of free rosiglitazone was obtained (yield 90%). X-ray powder diffraction examination (FIG. 1 as shown in the accompanied drawings) confirmed the amorphous nature of the product.

### Example 13

### Preparation of Amorphous Form of [(±)5-[[2-(5-ethyl-2-pyridinyl)ethoxyl]phenyl]methyl]-thiazolidine-2,4-dione (Free Pioglitazone)

Crystalline material of free pioglitazone (500 mg, prepared according to Example 1 of U.S. Pat. No. 4,687,777) was dissolved in methanol (100 ml) under stirring at 50° C., and the solution is heated to boiling (about 70 to 75° C.) until the material goes into solution. The solvent was completely distilled off under reduced pressure at 60° C. to 70° C. and the solid residue was isolated and dried in vacuum at 40° C. Thus 460 mg of amorphous form of free pioglitazone is obtained (yield 92%). X-ray powder diffraction examination (FIG. 2 as shown in the accompanied drawings) confirmed the amorphous nature of the product.

### Example 14

### Preparation of Amorphous Form of (±)5-[4-[2-(N-methyl-N-(2-pyridyl)amino) ethoxy]benzyl]thiazolidine-2,4-dione (Free Rosiglitazone)

Crystalline material of free rosiglitazone (500 mg, prepared according to Example 30 of U.S. Pat. No. 5,002,953) was dissolved in ethanol (100 ml) under stirring at 50° C., and the solution is heated to boiling (about 70° C. to 80° C.) until the material goes into solution. The solvent was completely distilled off under reduced pressure at 70° C. to 75° C. and the solid residue was isolated and dried in vacuum at 40° C. Thus about 0.45 g of amorphous from of free rosiglitazone is obtained (yield ˜90%). X-ray powder diffraction examination (FIG. 1 as shown in the accompanied drawings) confirmed the amorphous nature of the product.

### Example 15

### Preparation of Amorphous Form of [(±)5-[[2-(5-ethyl-2-pyridinyl)ethoxyl]phenyl]methyl]-thiazolidine-2,4-dione (Free Pioglitazone)

Crystalline material of free pioglitazone (400 mg, prepared according to Example 1 of U.S. Pat. No. 4,687,777) was dissolved in ethanol (80 ml) under stirring at 50° C., and the solution is heated to boiling (about 70 to 80° C.) until the material goes into solution. The solvent was completely distilled off under reduced pressure at 65° C. to 75° C. and the solid residue was isolated and dried in vacuum at 45° C. Thus 0.38 g of amorphous form of free pioglitazone is obtained (yield 95%). X-ray powder diffraction examination (FIG. 2 as shown in the accompanied drawings) confirmed the amorphous nature of the product.

### Example 16

### Preparation of Amorphous Form of (±)5-[4-[2-(N-methyl-N-(2-pyridyl)amino) ethoxy]benzyl]thiazolidine-2,4-dione (Free Rosiglitazone)

Crystalline material of free rosiglitazone (500 mg, prepared according to Example 30 of U.S. Pat. No. 5,002,953) was dissolved in acetonitrile (100 ml) under stirring at 50° C., and the solution is heated to boiling (about 60 to 70° C.) until the material goes into solution. The solvent was completely distilled off under reduced pressure at 55° C. to 65° C. and the solid residue was isolated and dried in vacuum at 45° C. Thus 0.45 g of amorphous from of free rosiglitazone is obtained (yield 90%). X-ray powder diffraction examination (FIG. 1 as shown in the accompanied drawings) confirmed the amorphous nature of the product.

### Example 17

### Preparation of Amorphous Form of [(±)5-[[2-(5-ethyl-2-pyridinyl)ethoxyl]phenyl]methyl]-thiazolidine-2,4-dione (Free Pioglitazone)

Crystalline material of free pioglitazone (500 mg, prepared according to Example 1 of U.S. Pat. No. 4,687,777) was dissolved in acetonitrile (80 ml) under stirring at 50° C., and the solution is heated to boiling (about 60° C. to 70° C.) until the material goes into solution. The solvent was completely distilled off under reduced pressure at 60° C. to 65° C. and the solid residue was isolated and dried in vacuum at 45° C. Thus 0.45 g of amorphous form of free pioglitazone is obtained (yield 90%). X-ray powder diffraction examination (FIG. 2 as shown in the accompanied drawings) confirmed the amorphous nature of the product.

While there has been described what is presently believed to be the preferred embodiments of the present invention, other and further modifications and change may be made without departing from the spirit of the invention. I intend to include all further and other modifications and changes which come within the scope of the invention as set forth in the claims.

I claim:

1. A pharmaceutical composition comprising an antidiabetic agent and a pharmaceutically acceptable stabilizer in an effective stabilizing amount, in which the composition retains at least about 95% w/w of the initial potency of antidiabetic agent, and contains not more than 0.5% w/w single largest degradant and not more than 1.5% w/w total degradants after storage for 3 months at about 40° C. and 75% relative humidity, and in which the stabilizers are selected from the group consisting of ascorbic acid, maleic acid, citric acid, malic acid, fumaric acid, or tartaric acid.

2. The pharmaceutical composition according to claim 1, wherein said antidiabetic agent is selected from the group consisting of (±)5-[[2-(5-ethyl-2-pyridinyl) ethoxyl]phenyl]methyl]-thiazolidine-2,4-dione (free pioglitazone) and (±)5-[4-[2-(N-methyl-N-(2-pyridyl)amino)ethoxy]benzyl]thiazolidine-2,4-dione (free rosiglitazone).

3. The pharmaceutical composition according to claims 1 and 2, wherein said antidiabetic agent is (±)5-[[2-(5-ethyl-2-pyridinyl) ethoxyl]phenyl]methyl]-thiazolidine-2,4-dione (free pioglitazone).

4. The pharmaceutical composition according to claims 1 and 2, wherein said antidiabetic agent is (±)5-[4-[2-(N-methyl-N-(2-pyridyl)amino)ethoxy]benzyl]thiazolidine-2,4-dione (free rosiglitazone).

5. The pharmaceutical composition according to claims 1, 2, or 4, wherein said free rosiglitazone is an amorphous form of free rosiglitazone.

6. The pharmaceutical composition according to claims 1, 2, or 4, wherein said free rosiglitazone is a crystalline form of free rosiglitazone.

7. The pharmaceutical composition according to claims 1, 2, or 3, wherein said free pioglitazone is an amorphous form of free pioglitazone.

8. The pharmaceutical composition according to claims 1, 2, or 3, wherein said free pioglitazone is a crystalline form of free pioglitazone.

9. The pharmaceutical composition according to claims 1, 2, or 4, wherein the amount of stabilizer is 15%-75% of the weight of free rosiglitazone in the composition.

10. The pharmaceutical composition according to claims 1, 2, or 3, wherein the amount of stabilizer is 15%-75% of the weight of free pioglitazone in the composition.

11. The pharmaceutical composition according to claims 1, 2, or 4, wherein the amount of stabilizer is 25%-60% of the weight of free rosiglitazone in the composition.

12. The pharmaceutical composition according to claims 1, 2, or 3, wherein the amount of stabilizer is 25%-60% of the weight of free pioglitazone in the composition.

13. A tablet or capsule containing a pharmaceutical composition according to claims 1, 2, 4, 5, or 6, wherein the amount of free rosiglitazone is 1 mg to 15 mg.

14. A tablet or capsule containing a pharmaceutical composition according to claims 1, 2, 4, 5, or 6, wherein the amount of free rosiglitazone is 1 mg, 2 mg, 4 mg, 8 mg, or 10 mg.

15. A tablet or capsule containing a pharmaceutical composition according to claims 1, 2, 3, 7, or 8, wherein the amount of free pioglitazone is 10 mg to 60 mg.

16. A tablet or capsule containing a pharmaceutical composition according to claims 1, 2, 3, 7, or 8, wherein the amount of free pioglitazone is 7.5 mg, 15 mg, 30 mg, or 45 mg.

17. A process of stabilizing antidiabetic agent in a solid pharmaceutical composition so that at least 95% w/w of the initial potency of antidiabetic agent is retained in the undegraded form, and not more than 0.5% w/w single largest degradant or not more than 1.5% w/w total degradant are formed after storage for three (3) months at about 40° C. and 75% relative humidity, wherein said process comprises mixing antidiabetic agent with a stabilizer by wet granulation or a dry method, the stabilizer being selected from the group consisting of ascorbic acid, maleic acid, malic acid, fumaric acid, citric acid, or tartaric acid.

18. A process according to claim 17, wherein said antidiabetic agent is selected from the group consisting of free rosiglitazone and free pioglitazone.

19. A compound which is an amorphous form of (±)5-[4-[2-(N-methyl-N-(2-pyridyl)amino)ethoxy]benzyl]thiazolidine-2,4-dione (free rosiglitazone).

20. The compound of claim 19 having substantially the same X-ray diffraction pattern as shown in FIG. 1.

21. A compound which is an amorphous form of (±)5-[[2-(5-ethyl-2-pyridinyl) ethoxyl]phenyl]methyl]-thiazolidine-2,4-dione (free pioglitazone).

22. The compound of claim 21 having substantially the same X-ray diffraction pattern as shown in FIG. 2.

23. A process to prepare amorphous form of antidiabetic agent comprising the steps of dissolving antidiabetic agent in a solvent to form a solution, and removing the solvent from the solution, and isolating the solid residue to afford amorphous form of antidiabetic agent.

24. The process of claim 23, wherein said antidiabetic agent is selected from the group consisting of free rosiglitazone and free pioglitazone.

25. The process of claim 23, wherein said antidiabetic agent is free rosiglitazone.

26. The process of claim 23, wherein said antidiabetic agent is free pioglitazone.

27. The process of claim 23, wherein said solvent is selected from the group consisting of tetrahydrofuran, acetonitrile, methanol, ethanol, isopropyl alcohol, n-butyl alcohol, t-butyl alcohol and mixture thereof.

28. The process of claim 23, wherein the solvent is methanol.

29. The process of claim 23, wherein the solvent is acetonitrile.

30. The process of claim 23, wherein the solvent is tetrahydrofuran.

31. A method for the treatment and/or prophylaxis of diabetes mellitus, conditions associated with diabetes mellitus and complication thereof, which comprise administering said pharmaceutical composition according to claims 1, 2, 3, or 4, to a human or non-human mammal in need of said treatment or prophylaxis.

32. The method of claim 31, wherein said method further comprises administering said antidiabetic agent of claims 2, 3, or 4 in combination with metformin hydrochloride.
